(19) United States
(12) Patent Application Publication
(10) Pub. No.: US 2026/0306399 A1
(43) Pub. Date: Oct. 8, 2026

(54) A CONSUMABLE GEL FOR HEALTH INGREDIENTS

(71) Applicant: Gelteq Limited, Caulfield (AU)

(72) Inventors: Nathan Jacob Givoni, Caulfield (AU); Julie Huynh, Caulfield (AU); Paul Michael Wynne, Caulfield (AU)

(21) Appl. No.: 19/100,703

(22) PCT Filed: Aug. 2, 2023
(86) PCT No.: PCT/IB2023/057834

§ 371 Date: Feb. 3, 2025

(30) Foreign Application Priority Data
Aug. 3, 2022 (GB) 2211290.8

(51) Int. Cl. A61K 9/00; A61K 9/06; A61K 47/12; A61K 47/36; A61K 47/44

(52) U.S. Cl. CPC A61K 9/0053; A61K 9/06; A61K 47/12; A61K 47/36; A61K 47/44

(57) ABSTRACT

An oral dosage form is disclosed. The dosage form includes an ingredient requiring taste masking, a first gelling agent, which is an agarose-based polymer gelling agent present in concentrations of between 0.001 g (1 mg) and 0.03 g (30 mg) per gram of water which concentration ensures hydration of, and the formation of, a firm gel with a bloom grade of between 100 and 250, and which does not exhibit plastic behaviour upon stress, but rather behaves elastically upon stress, to a failure point where the structure of the gel breaks or shears to irreversibly create new interfaces, making it more fluid when ingested, such that it is a drinkable gel. An oil/water emulsion including at least one lipid-based carrier, water, and an emulsifier. The oil/water emulsion comprises at least 14% by weight of the lipid-based carrier, based on the water content.

### CROSS-REFERENCE TO RELATED APPLICATIONS

This application claims priority to International Patent Application No. PCT/IB2023/057834 filed Aug. 2, 2023, which also claims priority to Great Britain Patent Application No. GB 2211290.8 filed Aug. 3, 2022, the contents of each of which is hereby incorporated by reference in its entirety.

### TECHNICAL FIELD

This invention relates to a dosage form comprising one or more health ingredients, particularly, but not exclusively, dietary, nutritional or pharmaceutical ingredients.

The dosage form is a stable, shearable, gel capable of delivering, particularly, multiple health ingredients in a single dosage form including at least one ingredient that requires taste masking, such as exemplified by bergamot, a bitter tasting ingredient. The dosage form may be used in human or animal health.

The dosage form is, particularly, but not exclusively, capable of delivering high doses of dietary, nutritional or pharmaceutical ingredients including: water soluble, water insoluble and/or lipid soluble ingredients, in a single delivery vehicle which would not readily lend themselves to delivery via tablets or capsules. By “high” is meant at least 1 g of one or more health ingredients, although the at least one ingredient that requires taste masking may be present in amounts of from 0.1 g, through 0.25 g and 0.5 g to 1 g or more.

Taste masking is achieved through the use of a high concentration oil/water emulsion which is stabilised in a shearable gel and which releases its' load when sheared, the oil elusion droplets providing a temporary coating on the tongue blunting the taste of the ingredient requiring masking.

By “high concentration” oil/water emulsion is meant at least or over 14% by weight of the lipid based carrier as compared to the water content of the dosage form.

### BACKGROUND

One of the most important factors in successfully developing health ingredients, particularly dietary, nutritional or pharmaceutical ingredients is ensuring patient compliance.

Currently, multiple product formats are used to deliver each of multiple e.g. nutritional or other health, e.g. active pharmaceutical ingredients (API's), products. The varied nature and number of such products leads to significant variability in compliance.

These health products, dietary, nutritional or pharmaceutical products, may be preventative or curative in nature. They include: prescription products, over-the-counter (OTC) products, and nutraceuticals and dietary supplements including, for example, products containing: vitamins, minerals, elements, proteins, carbohydrates, lipids, extracts of plant origin e.g. traditional Chinese medicines (TCM) or other indigenous medicines etc, either alone or in combination.

Poor compliance may be a consequence of a need to take e.g. multiple tablets, capsules or other dosage forms e.g. a tablet containing multiple vitamins, a capsule containing omega 3, a drink containing soluble fibre etc.

Tablets and capsules may prove difficult to take (swallow), particularly for certain patient groups, such as children and the elderly. For large dose products, or difficult to formulate products, or combination products, large tablet size and multiple tablet doses create compliance issues.

Other problems which effect patient compliance include the taste, smell or organoleptic feel of a product. The organoleptic properties may relate to aspects of product taste, product consistency, product texture, product mouth feel, longevity of taste and any other organoleptic sensation. Ingredients with unpleasant tastes are particular challenging and the focus of the claimed invention addresses products including such ingredient(s).

Some products require a separate drink to facilitate swallowing or to wash residues from the mouth, or a separate solid composition to be co-administered.

Examples include: replacement sugar products for hypoglycaemia, and whey protein, which is poorly dispersed in liquids, and is additionally required in large volumes.

Also, a delivery device may be required, such as a spoon, a syringe, or some other metering device to aid consumption and dose delivery. The requirement of such an additional device may adversely impact on compliance.

Other factors influencing effectiveness include the packaging, the chemical or physical stability of the composition, the bioavailability of the components, the visual appeal, and convenience of delivery.

Alternative dosage forms to capsules and tablets include drinks or gels comprising liquids or semi-solids.

“Energy gels” are typically highly viscous pastes containing thickeners, such as, xanthan gum. However, because they do not set, these products leave tongue residues that linger, may be unpleasant, and can irritate the oropharyngeal area, which may lead to coughing or choking.

These highly viscous pastes or gel products are those that will flow but tend to flow slowly, with a high internal resistance and a consistency like honey or ketchup. These known highly viscous pastes may be Newtonian or non-Newtonian fluids and may be shear thinning or shear thickening in nature. However, they all share the property that despite a degree of resistance, they plastically deform under stress—even where such behaviour is also known to have a threshold, such as Bingham plastic behaviour. Typically, these viscous pastes have a viscosity measurable between about 5000 and about 50,000 cP.

The term “set”, as used herein refers to jelly materials that set, and do not exhibit plastic behaviour upon stress, but rather behave elastically upon stress, to a failure point where the structure of the jelly breaks to irreversibly create new interfaces.

Applicant addressed this general problem in WO2019/215641 by developing a base gel comprising two different gelling agents:
a primary gelling agent which was an agarose-based polymer gelling agent, such as, an agar, and
a polyvalent ion gelled polymer which was an alginate-based polymer gelling agent, such as, but not limited to calcium alginate.

The details disclosed therein are incorporated by reference. The agarose-based polymer provides the key functional characteristics, namely it forms, in the amounts provided, a firm hydrated gel, with a bloom grade of between 100 and 250, which does not exhibit plastic behaviour upon stress, but rather behaves elastically upon stress, to a failure point where the structure of the gel breaks or shears irreversibly releasing water and creating new interfaces, making it more fluid when ingested, such that it is a drinkable gel.

The specification teaches forming emulsions incorporating up to 10% by weight of an oil or lipophilic components based on the water content of the dosage form.

This application addresses a specific challenge of taste masking in agarose-based polymer gels (which can be sheared to release water) and incorporate oils. Simultaneously, it needed to ensure the gels remained stable and functional. It has been designed to deliver one or more ingredients with an unpleasant taste, which ingredient(s) may be present in an amount, by weight, of from 0.1 g upwards.

### SUMMARY

In accordance with a first aspect of the invention there is provided an oral dosage form comprising
an ingredient requiring taste masking,
a first gelling agent, which is an agarose-based polymer gelling agent present in concentrations of between 0.001 g (1 mg) and 0.03 g (30 mg) per gram of water which concentration ensures hydration of, and the formation of, a firm gel with a bloom grade of between 100 and 250, and which does not exhibit plastic behaviour upon stress, but rather behaves elastically upon stress, to a failure point where the structure of the gel breaks or shears to irreversibly create new interfaces, making it more fluid when ingested, such that it is a drinkable gel; and
an oil/water emulsion comprising:
   at least one lipid-based carrier;
   water; and
   an emulsifier,
wherein the oil/water emulsion comprises at least 14% by weight of the lipid based carrier, based on the water content.

More typically the first gelling agent, which is an agarose-based polymer gelling agent is present in concentrations of between 0.001 g (1 mg) and 0.02 g (20 mg) per gram of water

The premise of the invention is based around ensuring droplets of emulsified oil are released from the gel when it shears such that they temporarily coat the tongue, blunting the taste of any ingredient(s) incorporated in the gel which are unpleasant (to receptors on the tongue).

Thus, the invention requires sufficient lipid-based carrier (oil) and emulsifier to achieve its' function as an effective taste masker whilst retaining the stability of the gel.

By stable is meant there is no phase separation of the oil and water components of the emulsion, and that the ingredient(s) did not precipitate out.

Selecting an oil that also provides desirable organoleptic properties was also required.

Any suitable oil including vegetable oils, seed oils, and marine oils can be used together with blends thereof.

Preferred oils were olive oil, sunflower oil, canola oil, coconut oil and oils containing medium chain triglycerides. Olive oil was particularly favoured for its neutral and organoleptic properties.

In contrast to the invention disclosed in WO2019/215641 the invention can be used with a single agarose based gelling agent, and it is the greater concentration of the emulsified oil that provides the effective taste masking—since it is the release of the emulsified oil on shearing that provides a “protective” (taste blunting) coating to the tongue.

Agar proved particularly beneficial in providing enhanced stability.

Applicant has determined that the optimum concentration of emulsified oil (by weight) is between 14% and 28% by weight of oil to water, although Applicant has determined that it is possible, in some circumstances, to get as much as 30% by wt of oil to water.

More preferably, the emulsified oil comprises at least 20% by weight the lipid based carrier.

The incorporation of an emulsifier further improves performance. A preferred emulsifier is one of lecithin, polyethylene glycol (PEG) and Polysorbate 80.

The emulsifier should be present in an amount of from 0.1% to as much as 6.0% by weight. A preferred range is from 3% to 5%. Many other benefits of these shearable gels are as disclosed in WO2019/215641.

A firm gel is a “set” gel or jelly characterised in an industrial and scientific context by the Bloom test, which is used to provide a relative firmness and strength of these gels. This test originated in 1925 by Oscar T. Bloom. The test determines the weight in grams needed by a specified plunger (normally with a diameter of 0.5 inch (11 mm) to depress the surface of the gel by 4 mm, without irreversibly breaking the gel to create new interfaces, at a specified temperature. This result is the Bloom grade. A higher number indicates a stiffer product. For example, gelatin used in food usually runs from 125 Bloom to 250 Bloom.

For the purpose of this document the terms gel and jelly are interchangeable.

A set gel or jelly is somewhat elastic which means that under stress they deform, but when that stress is released they substantially return back to their original form. For example, if gently pressed by the underside of a spoon, a set jelly will be pushed down, but when the spoon is removed the original surface returns to its original position. It is not plastic, whereby under the same stress the surface would be deformed and then remain deformed once the pressure is removed. Set gels or jellys, rather than deform plastically, will fail by breaking irreversibly to create a new interface if the stress is further increased beyond its failure point.

The oral dosage form of the invention may typically be provided in a volume of between 5 ml and 250 ml, depending on use, and is delivered from a container. For small containers (5-25 ml) this may take the form of a sachet or straw, but for larger containers (25 to 250 ml), this typically takes the form of a pouch, with a spout or straw, such that a sucking action shears the gel, such that it becomes more fluid with new interfaces being created, sometimes with the release of some water and the emulsion.

It is the shearing and creation of new interfaces, and the concominent release of the emulsion which has the advantage that it facilitates powerful taste masking.

The agarose-based polymer gelling agent, is preferably, an agar.

The agarose-based polymer gelling agent also advantageously behaves as an emulsifier so that additional emulsifiers are required in lesser amounts.

A polyvalent ion gelled polymer, when used, is most preferably an alginate-based polymer gelling agent, such as, but not limited to calcium alginate. Alternatives include, guar gum, gellan gum, xanthan gum, pectin and kudzu.

A particularly favoured combination of gelling agents comprise agar as the primary gelling agent and calcium alginate as the secondary gelling agent. This combination proved particularly versatile allowing combinations of two or more of water soluble, water insoluble and/or lipid-based ingredients to be formulated together in a single dosage form.

These two fundamentally different gelling agents provide a set gel structure via different interaction mechanisms. The advantage of the combination is its versatility as a base, as the combination overcome scenarios where gelation would otherwise by inhibited by the presence of different ingredients.

Agar consists of a mixture of two polysaccharides: agarose and agaropectin, with agarose making up about 70% of the mixture. Agarose is a linear polymer, made up of repeating units of agarobiose, a disaccharide made up of D-galactose and 3,6-anhydro-L-galactopyranose. Agaropectin is a heterogeneous mixture of smaller molecules that occur in lesser amounts and is made up of alternating units of D-galactose and L-galactose heavily modified with acidic side-groups, such as sulphate and pyruvate. Any known variant of Agar may be used in the invention.

Agar exhibits hysteresis, melting at 85° C. and solidifying at from 32° C. to 40° C. This property lends a suitable balance between easy melting and good gel stability, at relatively high temperatures.

The gelling also helps provide stability, at room temperature (and above), meaning cold storage can be avoided.

The most common alginate used in food is sodium alginate. In order to form a gel, sodium alginate needs to come into contact with divalent ions, such as calcium (Ca2+). When sodium alginate is added to a solution of a calcium salt, such as the chloride or citrate, a gel forms as the sodium ions (Na+) are exchanged with calcium ions (Ca2+) and the polymers become crosslinked. The divalent ions, such as calcium, are able to form crosslinks between alginate polymers because they can form two bonds, whereas monovalent ions, such as sodium, can only form one bond. The longer the alginate is in contact with the divalent ion solution, the more rigid the gel will become, as more crosslinks are formed. Also, depending on the concentration of divalent ions, the gels are either thermo-reversible (at low concentrations) or thermos-irreversible (at high concentrations).

Alginic acid is a linear copolymer with homo-polymeric blocks of (1-4)-linked β-D-mannuronate (M), and its C-5 epimer, α-L-guluronate (G), which are covalently linked together in different sequences or blocks.

Sodium alginate is the sodium salt of alginic acid. Its empirical formula is NaC₆H₇O₆. Sodium alginate is a gum, extracted from the cell walls of brown algae.

Potassium alginate is a chemical compound that is the potassium salt of alginic acid. Its empirical chemical formula is KC₆H₇O₆.

Calcium alginate, made from sodium alginate from which the sodium ion has been removed and replaced with calcium, has the chemical formula C₁₂H₁₄CaO₁₂.

The gel formulation of the invention can be used to deliver unpleasant tasting ingredients, including those which are:
i) Water soluble;
ii) Water insoluble; or
iii) Lipids

Typically, but not essentially, a dose including upwards of 0.1 g of at least one ingredient requiring taste masking and optionally, between 1 g and 40 g of additional ingredients is delivered in a volume of between 5 and 45 ml of gel. These ingredients can include other nutraceutical or health ingredients.

The base gel formulation can be used to load a single ingredient requiring taste masking, but the format makes it particularly effective for delivering multiple health ingredients, including the ingredient requiring taste masking, in a simple to take, simple to use dosage form which can retain ingredients which would typically be difficult to formulate in a single dosage form due to their different properties and can be administered orally by squeezing or sucking the gel from its container.

A single ingredient requiring taste masking, is most likely to be delivered in a smaller volume format (5 ml to 25 ml), whereas a larger format (25 ml to 250 ml) is preferred where multiple health ingredients, particularly those required in large doses, and are required alongside the ingredient(s) requiring taste masking.

Such multiple health ingredients may include, for example, proteins, carbohydrates, dietary fats, vitamins and minerals.

The Proteins optionally include: milk protein concentrate, whey protein concentrate, soya protein isolate, and sodium caseinate which may additionally include Amino Acids such as one or more of: L-Glutamine, L-Glycine, L-Leucine, L-Isoleucine, L-Valine, Beta Alanine, L-Arginine, and Taurine.

The Carbohydrates optionally include, for example: maltodextrin, inulin, fructose, and glucose syrup.

The Dietary Fats optionally include: vegetable fats and oils, fish oils, coconut oil, mono, di and triglycerides of fatty acids, and lecithin.

The Vitamins optionally include: Vitamin C (Ascorbic Acid), Vitamin K (Menaquinone-7), Vitamin E (dl-Alpha Tocopheryl Acetate), Niacin (Niacinamide), Vitamin A (Retinyl Acetate), Pantothenic Acid (Calcium D-Pantothenate), D-Biotin, Folic Acid, Vitamin D (Cholecalciferol), Vitamin B6 (Pyridoxine Hydrochloride), Riboflavin (Riboflavin 5 Phosphate Sodium), Thiamin (Thiamin Hydrochloride), Vitamin B12 (Cyanocobalamin), Creatine Monohydrate, beta carotene, lutein, lycopene oleoresin, choline chloride, L-carnitine, nicotinamide, calcium D-pantothenate, riboflavin, pyridoxine hydrochloride, pteroylmonoglutamic acid, phytomenadione, resveratrol, Alpha Ketoglutarate, Calcium Hydroxy Methylbutyrate (HMB), Tricreatine Malate.

The Minerals optionally include Essential and Trace Mineral sources and salts such as one or more of: Tri-potassium Citrate, Sodium Chloride, Trisodium Citrate, Magnesium Oxide, Zinc Gluconate, Iron Fumarate, Manganese Gluconate, Copper Gluconate, Potassium Iodide, Sodium Molybdate, Chromium Picolinate, sodium selenite, chromium chloride, potassium iodide, sodium fluoride, sodium chloride, potassium chloride, calcium hydroxide, sodium ascorbate, magnesium hydroxide potassium hydroxide, ferrous lactate, zinc sulphate, manganese sulphate, and Phosphates such as one or more of sodium triphosphate, tricalcium phosphate, potassium phosphate.

Some or all of these may be incorporated into the emulsion.

### Other Components May Include:

Antioxidants, such as one or more of: ascorbyl palmitate, vitamin C (ascorbic acid) and vitamin E (d-alpha tocopherol).

A pH modifier, such as one or more of: citric acid, and ascorbic acid, being used in amounts to provide a pH of from 4 to 7.

Preservatives, such as sorbic acid. or a salt thereof e.g. potassium sorbate

In one embodiment the gel of the invention is used to deliver ingredients in the form of an entrapped stable emulsion.

The lipid component, for example, sunflower or olive oil, is emulsified with the water, usually with stirring, and then caused to gel with the agar. Additional emulsifying agents may be added to facilitate the emulsification prior to gelling.

Typical food grade or pharmaceutical grade emulsifiers, such as, for example a lecithin, esters of monoglycerides of fatty acids and mono, di and tri glycerides of fatty acids are added.

The provision of multi-phase combinations can be accommodated. For example, an ingredient may be more stable in an oil in water emulsion. The set gel formed can trap & immobilise multiple phases enhancing stability as well as e.g. masking taste. The set gel is easily sheared during delivery to form new wet interfaces thereby facilitating delivery and taste masking through the release of the emulsion.

In its stored state the formulation has a firm jelly form, is stable at room temperature, but when sucked or otherwise subjected to a gentle force, shears to create new wet interfaces which facilitate swallowing. Swallowing is eased because the gel interfaces created are slippery because they are inherently wet, due to the water within the structure.

This novel formulation provides a chemically and physically stable matrix which can be used to entrap water soluble components, non-water soluble lipids, and lipid soluble components alike and provide effective taste masking.

As a result of the entrapment, there is a reduced surface contact to taste buds but when sheared the emulsion is released coating the tongue thereby blunting taste. Unlike viscous paste gels, the gels of the present invention provide minimal residues, other than displaced water, and are very much easier to swallow as a set jelly will shear on sucking in contrast to alternative solid/semi-solid formats that are plastic (not elastic).

Advantageously, because they shear, they release components quickly, even in high doses, and the released emulsion provides additional taste masking.

Unlike tablets or capsules the platform is highly flexible in allowing different health ingredients to be provided in combinations in a single dosage form which has benefits over the alternatives currently on offer.

The additional use of an alginate means that some beneficial calcium salts can be incorporated as part of the gelling process. Thus, for formulations directed towards bone health Calcium and/or Magnesium salts (e.g. calcium citrate or carbonate) will aid gelation by complexing. Calcium citrate may also enhance flavour.

Solid suspensions may include nano-milled materials, such as, curcumin or glucosamine in a format which may enhance their bioavailability.

Agar provides clear gels, without coloured specks. However, Agar and alginate gels are also visually very clear, but more importantly the alginate cross links provide a more structured gel with a different gelling mechanism to the carbohydrate non-ionic gelled polymers improving versatility and performance.

However, adding Ca, Mg and Zn salts, such as chloride or citrate, interferes with the setting of Agar and some other gum polymers. Thus, to obtain good setting results Agar and Alginate should be used in particular ratios (w/w). The preferred ratios favour an excess of agar to alginate, and thus a ratio of between 8:1 to greater than 1:1, more preferably 5:1 to greater than 1:1 and most preferably about 3: greater than 1 Agar to Alginate is preferred. The Agar acts as an emulsifier and gelling agent, whilst the alginate together with the metal 2+ ions (Ca2+, Mg2+, Zn2+) form a structured gel counteracting the effect of these ions where only Agar is present.

Agars and alginates are both suitable for vegans, vegetarians and most religious diets whereas gelatine is not, as it's an animal protein, usually obtained from pigs.

The advantage of combining agar and alginate gels is that they are less sensitive to temperature effects than other gelling agents. They are also less affected by components that may inhibit gelation, such as oils, changes in pH, and high concentrations of complex carbohydrates, such as, maltodextrins. Agar and alginate gels also tend to be faster setting, which is an advantage in manufacturing, facilitating faster and more robust production.

The lipids used in the practising of the invention included any suitable food grade oil of animal or plant origin. Non-limiting examples include: corn oil, olive oil, sunflower oil, and coconut oil. The lipid material may advantageously include lipid vitamins such as any of vitamin D or vitamin E.

### Other Preferred Features Include

A dosage form including one or more health ingredients which are water soluble ingredients and lipid soluble ingredients.

A dosage form wherein the one or more water soluble health ingredients are selected from the group comprising or consisting of water soluble vitamins, carbohydrates, sugars and amino acids.

A dosage form wherein the one or more lipid-soluble ingredients are selected from the group comprising or consisting of: oils, particularly fish and algal oils, DHA, EPA and lipid soluble vitamins D and E.

A dosage form comprising one or more of a protein, vitamin, mineral, trace elements and a plant or marine extracts.

A dosage form wherein the one or more health ingredients are present in an amount of at least 1 g.

A dosage form further comprising a second gelling agent which is a polyvalent ion gelled polymer gelling agent which is an alginate-based polymer gelling agent.

Preferably the first gelling agent is present in an amount greater than the second gelling agent and more preferably still the agarose-based polymer gelling agent and alginate-based polymer gelling agent are present in a ratio of from greater than 1:1 to 3:1.

Furthermore the dosage form is packaged in either:
a pouch with an opening for facilitating the drinking of the gel by squeezing and/or sucking;
a sachet or straw;
a syringe; or
   any other packaging facilitating delivery by sucking, squeezing or pushing the gel from the packaging.

In addition—compliance of animal species such as pets, farm animals, or other domesticated animals for veterinary products is a challenge, as it can be very challenging for animal carers to ensure animals consume oral delivery forms such as tablets or capsules or powders.

The benefits can also be provided to improve veterinary product compliance.

Benefits of the invention include:
Taste masking: The gels of the invention allows any mixture of nutrients and most poor, strong & unpleasant flavours—such as bitterness, sweetness, fish oils, curcumin (spice/peppery) to be masked.
Large volumes of nutrients may additionally be formulated in one easy to consume dosage form: Multiple ingredients and large doses can be combined in a single dosage form.
Overcomes choking risks posed by large tablets: Gels are much easier to swallow than pills. The format aids all swallowing issues, whether young children or the elderly or merely those who have a predisposed swallowing problem.
Stable at room temperature. The gel nature provides stability without the need for cold storage.
Includes water as an ingredient. The presence of “locked up” but “releasable” water also ensure hydration is achieved, even if released as an emulsion, and no additional fluids are required to enable the product to be swallowed. This also helps aid digestion and provides an improved mouth feel.

### BRIEF DESCRIPTION OF THE DRAWINGS

Embodiments of the invention are further described hereinafter with reference to the accompanying drawing, in which:

FIG. 1 is an illustration of a dispensing dosage form of the invention.

### DETAILED DESCRIPTION

The general process for the preparation of a gel formulation is set out in Example 1 below:

### Example 1

### Production Methodology

Any water-soluble ingredients are dissolved in water first. These may include a nutritional ingredient such as glucose, as well as preservatives and dilute acids (for pH control). An exemplary preservative is potassium sorbate, and an exemplary pH control agent, if required, is a weak acid, such as, citric acid, ascorbic acid, acetic acid or dilute HCl.

To facilitate efficient addition of the gelling agent(s), the solution is preferably stirred and heated to near boiling. The agar and optionally sodium alginate are added slowly, to allow for gradual hydration, with steady constant stirring, ensuring satisfactory dispersion or dissolution. The dispersion or solution is kept at or around boiling point for a time, between 4-5 minutes, sufficient to ensure proper hydration, especially of the agar.

A separate emulsion is formed mixing oil, water and any other ingredient to be incorporated into the emulsion.

The two components are then mixed and the finished product is filled into e.g. sachets and allowed to cool such that it forms a firm gel.

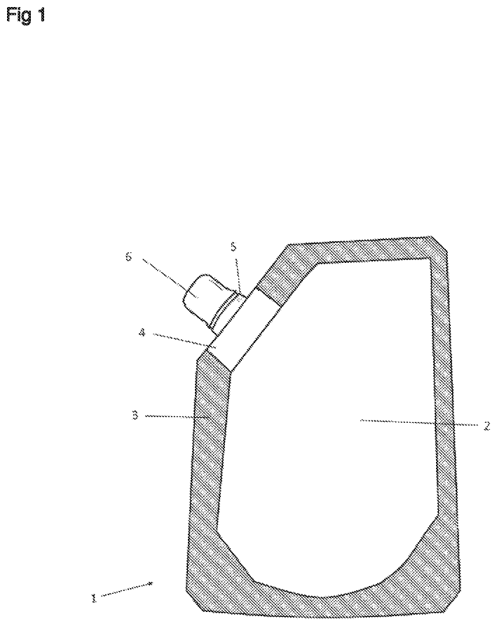

The sachet may be a simple sachet or tube for small volumes (5-25 ml) or for larger volumes (25 to 250 ml) a pouch, as illustrated in FIG. 1. The pouch (1) is formed from, for example, plastics or foil layers that are sealed together around an edge (3) incorporating a filling and dispensing aperture comprising a plug (4), spout (5) and cap (6). The pouch is filled through the mouth piece (5) and is secured with a cap (6) which may be provided with a frangible seal. In use the subject removes the cap and drinks the gel by squeezing it and/or sucking it from the pouch (1) through the spout (5).

Alternative packaging for delivery may include a sachet or straw; a syringe; or any other packaging facilitating delivery by sucking, squeezing or pushing the gel from the packaging.

When preparing the gel it is desirable to limit the heating period to minimise water loss from evaporation as excessive evaporation can lead to product variability.

Preferably the agar is used in concentrations of between 0.001 g (1 mg) and 0.02 g (20 mg) per gram of water. More preferably between 0.005 g (5 mg) and 0.01 mg (10 mg) per gram of water, and most preferably between 0.006 g (6 mg) and 0.009 g (9 mg) mg per gram (equivalent to 1 ml) of water.

If used, the alginate is used in concentrations of between 0.0005 g (0.05 mg) and 0.01 g (10 mg) per gram of water. More preferably between 0.001 g (1 mg) and (0.008 g (8 mg) per gram of water, and most preferably between 0.001 g (1 mg) and 0.005 g (5 mg) per gram of water.

With the alginate a source of divalent ions is added to enhance the alginate gel formation. Suitable ingredients include calcium, zinc and/or magnesium salts. For example, calcium citrate, magnesium citrate or zinc citrate are good forms of soluble salts and can also provide mineral supplementation. Other salts may however be used.

Favoured lipids for forming the emulsion are neutral oils such as olive oil and sunflower oil. Typically, the lipid level is added in concentrations of from 14 to 28%, but in certain circumstances up to 30%, w/w of the total water. The water component may be “split” and the gel and emulsion mixed.

A mechanical homogeniser, such as a Silverson homogeniser, is advantageously used to facilitate the formation of an emulsion. Any known food grade emulsifier may be added to improve the emulsion quality and physical stability, such as, for example, a lecithin.

Preferably the emulsifier is non-animal sourced, such as soya lecithin, rather than egg lecithin.

In an advantageous process the emulsion is, as noted above, made as a separate cool water mix precursor, and is combined with a separate hot agar in water precursor, (the water being split as appropriate) to speed cooling and gel formation.

Further components may be dissolved or dispersed into the gel or emulsion. Where a nutritional component is neither water or lipid soluble, this may be prepared as a suspension in either lipid or water. It is advantageous if this component is milled to a fine particle size, as this may improve suspension stability, and may also improve bioavailability. Advantageously the insoluble component may be milled in wet or dry conditions, and may be ball milled, or media milled or nano-milled or milled in a high shear homogeniser. The milling may be conducted in the presence of a milling aid, such as leucine, sodium lauryl sulphate, and metal stearates.

After heating, this liquid formulation can be metered hot into the desired containers and allowed to set as a gel.

More specifically for e.g. a product for hypoglycaemia, during the manufacturing processing the following are undertaken:

The gel production process involves the following:

Weigh the non-gelling agents (e.g. glucose, potassium sorbate, water) in appropriate scaled quantities and add them to a suitable mixing vessel, in the order of e.g. Glucose (or other carbohydrate) as required; potassium sorbate or other preservative as needed, and water.

Apply steady stirring, e.g. at lab scale this is a magnetic stirrer bar. Cover to minimise evaporation and water loss.

Pre-weigh the specified nutrients required for composition. Mix these non-gelling ingredients while applying heat to the vessel contents. Add one nutrient at a time to the vessel, and allow for it to be fully dispersed and dissolved, then add the next nutrient. When all the nutrients have been added, allow to mix for an additional 2-3 minutes.

Pre-weigh the gelling agent (agar agar).

Stir for 2-3 minutes to ensure all the agents that have been incorporated and are no longer visible. Where insoluble components are used, incorporate using a homogeniser.

Mechanically form an emulsion with the lipids, some of the water and at least the ingredient(s) to be taste masked using a homogeniser, heating as appropriate.

Add the emulsion to the gel fraction and allow the formulation to cool and gel.

The Bloom test is used to characterise the resulting grade of the set gel. Preferably, a Bloom grade of at least 100 through 110, 120, 130 and 140 is obtained.

Preferably, a Bloom grade of less than 250, less than 220, less than 200 to less than 180 is obtained.

An optimal Bloom grade of between 140 and 180 is desired.

### Example 2

### Development and Testing

A preferred base gel, into which a plurality of ingredients promoting health or well-being (not shown) are added, particularly nutritional ingredients, comprises agar because of its water releasing ability.

In WO2019/215641, Example 1, it is taught that lipid may be added to the formulations described therein in amounts up to 10% w/w of the water (i.e to 2.5 g in 25 ml of water.) Example 2 therein teaches a formulation with 0.5 g of omega 3 in 24 g of water (approx. 2%) and in Examples 4 to 5 the omega 3 level is 1 g in 24 g of water (approx. 4%).

Applicant found that with a bitter testing ingredient, such as bergamot, the oil and emulsifier, also present, did not adequately taste mask the product at such concentrations. They therefore undertook a series of experiments to see how they might better address the taste masking of ingredients in water releasing gels. Key test data is set out in the Examples below: Example 2a—Starting formulation (no emulsion)

Sample 1C - pH adjusted

| Ingredient | Required mass | Unit | pH |
|---|---|---|---|
| Bergamet 47% BPF | 1 | g | 4.41 |
| Water | 25 | g | |
| Potassium sorbate | 100 | mg | |
| Agar | 0.19 | g | |

In taste tests this product was very bitter.

Applicant therefore decided to incorporate the ingredient with an offensive taste profile (exemplified with bergamot) into an emulsion as per Example 2b.

### Example 2b (Sample 4C)

Sample 4C - Emulsion with low oil

| Ingredient | Required mass | Unit |
|---|---|---|
| Water | 17 | g |
| Potassium sorbate | 100 | mg |
| Lecithin | 0.2 | g |
| Water | 8 | g |
| Sunflower oil | 0.08 | g |
| Bergamet 47% BPF | 1 | g |
| Agar | 0.24 | g |

This product, in which the oil in water emulsion had a concentration of 0.3% w/w with water (0.08/25×100) did not improve the taste profile.

In Examples 2c through 2f they increased the oil level in stages through 1.5%, 4.5%, 7.5% to a maximum of 10.5%.

### Example 2c (Sample 4d)

Sample - Emulsion with increasing oil

| Ingredient | Required mass | Unit |
|---|---|---|
| Water | 17 | g |
| Potassium sorbate | 100 | mg |
| Lecithin | 0.2 | g |
| Water | 8 | g |
| Sunflower oil | 0.4 | g |
| Bergamet 47% BPF | 1 | g |
| Agar | 0.24 | g |

### Example 2d

Sample - Emulsion with increasing oil

| Ingredient | Required mass | Unit |
|---|---|---|
| Water | 17 | g |
| Potassium sorbate | 100 | mg |
| Lecithin | 0.2 | g |
| Water | 8 | g |
| Sunflower oil | 1.2 | g |
| Bergamet 47% BPF | 1 | g |
| Agar | 0.24 | g |

### Example 2e

Sample - Emulsion with increasing oil

| Ingredient | Required mass | Unit |
|---|---|---|
| Water | 17 | g |
| Potassium sorbate | 100 | mg |
| Lecithin | 0.2 | g |
| Water | 8 | g |
| Sunflower oil | 2.0 | g |
| Bergamet 47% BPF | 1 | g |
| Agar | 0.24 | g |

### Example 2f

Sample 4f - Emulsion with increasing oil

| Ingredient | Required mass | Unit |
|---|---|---|
| Water | 17 | g |
| Potassium sorbate | 100 | mg |
| Lecithin | 0.2 | g |
| Water | 8 | g |
| Sunflower oil | 2.8 | g |
| Bergamet 47% BPF | 1 | g |
| Agar | 0.24 | g |

There was a noticeable improvement in taste at the highest concentrations, but the product was still considered unpalatable.

In Example 2g they explored incorporating Bergamot into variants of a ketogenic product they were developing (very high lipid content).

### Example 2g (5c)

Sample 5C - ½ Keto Emulsion

| Ingredient | Required mass | Unit |
|---|---|---|
| Bergamet 47% BPF | 1 | g |
| Water | 10 | g |
| Potassium sorbate | 100 | mg |
| Lecithin | 0.5 | g |
| Water | 11.5 | g |
| Sunflower oil | 3 | g |
| Agar | 0.24 | g |

This product, in which the oil in water emulsion had a concentration of 14% (3/21.5×100) did significantly improve the taste profile. The bitterness was reduced, but was still noticeable. Significantly, the immediate bitter taste disappeared very quickly. Thus, the higher concentration of lipid muted the bitterness.

Based on the above finding Applicant pushed the oil content even higher to determine the impact on both taste and stability and the results are illustrated in Examples 2h to 2j.

### Example 2h (16% Oil/Water Emulsion)

| Ingredient | Required Mass | Unit |
|---|---|---|
| Bergamet 47% BPF | 1.00 | g |
| Water | 10.00 | g |
| Potassium sorbate | 0.10 | g |
| Lecithin | 1.50 | g |
| Water | 15.00 | g |
| Olive oil | 4.00 | g |
| Agar | 0.26 | g |

A further improvement in taste was noted and the olive oil was considered a more palatable oil with better organoleptic properties than the sunflower oil.

### Example 2i (7b) (20% Oil/Water Emulsion)

| Ingredient | Required Mass | Unit |
|---|---|---|
| Bergamet 47% BPF | 1.00 | g |
| Water | 10.00 | g |
| Potassium sorbate | 0.10 | g |
| Lecithin | 1.50 | g |
| Water | 15.00 | g |
| Olive oil | 5.00 | g |
| Agar | 0.26 | g |

### Example 2j (7c) (24% Oil/Water Emulsion)

| Ingredient | Required Mass | Unit |
|---|---|---|
| Bergamet 47% BPF | 1.00 | g |
| Water | 10.00 | g |
| Potassium sorbate | 0.10 | g |
| Lecithin | 1.00 | g |
| Water | 15.00 | g |
| Olive oil | 6.00 | g |
| Agar | 0.28 | g |

Both Examples 2i and 2j were able to successfully taste mask the bitterness of bergamot.

They were also stable, by which is meant their was no phase separation of the oil and water components of the emulsion, and that the ingredient(s) did not precipitate out.

### CONCLUSION

From the tests conducted Applicant broadly concluded:
0.3% to 10.5% of a lipid based carrier gave a stable gel with a soft texture but the emulsion did not adequately taste mask bitterness/bergamot;
10.5% to 14% of lipid based carrier gave a stable gel with a firmer texture and the emulsion reduced bitterness. As the concentration increased there was a significant and noticeable improvement compared to the prior art oils;
14% to 28.0% (and up to 30% not shown) oil gave a stable gel with the firmest texture which very significantly removed the bitterness taste and additionally, where an unpleasant taste was perceived it was only short lived.

It will be appreciated that the skilled nutritionist will be able to formulate multiple variations of the above using their general skill and knowledge.

1. An oral dosage form comprising
an ingredient requiring taste masking;
a first gelling agent, which is an agarose-based polymer gelling agent present in concentrations of between 0.001 g (1 mg) and 0.03 g (30 mg) per gram of water which concentration ensures hydration of, and the formation of, a firm gel with a bloom grade of between 100 and 250, and which does not exhibit plastic behaviour upon stress, but rather behaves elastically upon stress, to a failure point where the structure of the gel breaks or shears to irreversibly create new interfaces, making it more fluid when ingested, such that it is a drinkable gel; and
an oil/water emulsion comprising:
at least one lipid-based carrier;
water; and
an emulsifier,
wherein the oil/water emulsion comprises at least 14% by weight of the lipid-based carrier, based on the water content.

2. A dosage form as claimed in claim 1 wherein the oil/water emulsion comprises at least 20% by weight of the lipid-based carrier, based on the water content.

3. A dosage form as claimed in claim 1 wherein the oil/water emulsion comprises a maximum of 30% by weight of the lipid-based carrier, based on the water content.

4. A dosage form as claimed in claim 1 wherein the agarose-based polymer gelling agent is agar.

5. A dosage form as claimed in claim 1 wherein the lipid-based carrier is selected from olive oil, sunflower oil, coconut oil and a medium chain triglyceride (MCT) oil, or bends including such oils.

6. A dosage form as claimed in claim 1 wherein the emulsifier is lecithin, polyethylene glycol (PEG) or Polysorbate 80.

7. A dosage form as claimed in claim 1 further comprising one or more preservatives.

8. A dosage form as claimed in claim 7 wherein the preservative is sorbic acid.

9. A dosage form as claimed in claim 1 further comprising a pH modifier.

10. A dosage form as claimed in claim 9 wherein the pH modifier is used in an amount to maintain the pH in the range of 4 to 7.

11. A dosage form as claimed in claim 1 further comprising one or more health ingredients in addition to the ingredient(s) requiring taste masking.

12. A dosage form as claimed in claim 11 wherein the one or more health ingredients is selected from water soluble ingredients and lipid soluble ingredients.

13. A dosage form as claimed in claim 12 wherein the one or more water-soluble health ingredients are selected from the group comprising or consisting of: water soluble vitamins, carbohydrates, sugars, and amino acids.

14. A dosage form as claimed in claim 12 wherein the one or more lipid-soluble ingredients are selected from the group comprising or consisting of: oils, particularly fish and algal oils, DHA, EPA and lipid soluble vitamins D and E.

15. A dosage form as claimed in claim 12 further comprising one or more of a protein, vitamin, mineral, trace elements and a plant or marine extracts.

16. A dosage form as claimed in claim 12 wherein the one or more health ingredients are present in an amount of at least 1 g.

17. A dosage form as claimed in claim 1 further comprising a second gelling agent which is a polyvalent ion gelled polymer gelling agent which is an alginate-based polymer gelling agent.

18. A dosage form as claimed in claim 17 wherein the first gelling agent is present in an amount greater than the second gelling agent.

19. A dosage form as claimed in claim 18 wherein the agarose-based polymer gelling agent and alginate-based polymer gelling agent are present in a ratio of from greater than 1:1 to 3:1.

20. A dosage form as claimed in claim 1 which is packaged in either:
a pouch with an opening for facilitating the drinking of the gel by squeezing and/or sucking;
a sachet or straw;
a syringe; or
any other packaging facilitating delivery by sucking, squeezing or pushing the gel from the packaging.
